(19) United States
(12) Patent Application Publication
(10) Pub. No.: US 2023/0372097 A1
(43) Pub. Date: Nov. 23, 2023

(54) Automated Balloon Inflation Device for Transcatheter Heart Valve Implantation

(71) Applicant: St. Jude Medical, Cardiology Division, Inc., St. Paul, MN (US)

(72) Inventors: Michael Shane Morrissey, St. Paul, MN (US); Peter J. Ness, Minneapolis, MN (US); Tyler Govek, Minneapolis, MN (US); Abhijeet Joshi, Irvine, CA (US); Neil Theisen, Champlin, MN (US)

(73) Assignee: St. Jude Medical, Cardiology Division, Inc., St. Paul, MN (US)

(21) Appl. No.: 18/311,458

(22) Filed: May 3, 2023

Related U.S. Application Data
(60) Provisional application No. 63/343,479, filed on May 18, 2022.

(51) Int. Cl. A61F 2/24

(52) U.S. Cl. CPC A61F 2/2433; A61F 2230/0069

(57) ABSTRACT

A method of implanting a prosthetic heart valve includes delivering the prosthetic heart valve to a native valve annulus while the prosthetic heart valve is crimped over a deflated balloon of a delivery device. The method may include advancing fluid through the delivery device and into the balloon to inflate the balloon and to expand the prosthetic heart valve into the native valve annulus. The method may include monitoring a pressure within the delivery device while advancing fluid through the delivery device. The method may also include displaying the monitored pressure on a display device in real time during monitoring the pressure.

### CROSS-REFERENCE TO RELATED APPLICATIONS

This application claims priority to U.S. Provisional Patent Application No. 63/343,479, filed May 18, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent' s interior or luminal surface, its exterior or abluminal surface, and/or on both surfaces. A cuff helps to ensure that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a manual syringe through the balloon catheter to cause the balloon to begin to fill and expand, and thus cause the overlying prosthetic heart valve to expand into the native annulus. The reliance on fully manual balloon inflation may not be optimal, and it may be desirable to have partial or complete automation of the balloon inflation process, for example to provide more consistent and predictable results of the balloon expansion. For example, the predictability of how a balloon expandable prosthetic heart valve expands can vary greatly depending on how quickly the user inflates the balloon. Such systems may also be able to assist in providing data that can be used during the procedure, and which may also be collected among many procedures to learn information relating to important parameters of the balloon inflation that may not be otherwise easily determined from the typical manual process. This information may be gathered and used to refine the partial or fully automated balloon expansion process for future procedures. It would also be desirable for balloon inflation systems (or accessory components thereof) to be able to reliably de-air the balloon catheter system prior to use, preferably with an objective mechanism (e.g. other than only by eyesight) by which to confirm that no more than an acceptable amount of air remains in the catheter prior to delivery.

### BRIEF SUMMARY OF THE DISCLOSURE

According to one aspect of the disclosure, a method of implanting a prosthetic heart valve includes delivering the prosthetic heart valve to a native valve annulus while the prosthetic heart valve is crimped over a deflated balloon of a delivery device. The method may include advancing fluid through the delivery device and into the balloon to inflate the balloon and to expand the prosthetic heart valve into the native valve annulus. The method may include monitoring a pressure within the delivery device while advancing fluid through the delivery device. The method may also include displaying the monitored pressure on a display device in real time during monitoring the pressure.

According to another aspect of the disclosure, a prosthetic heart valve delivery system may include a handle, a balloon catheter extending from the handle, and a balloon positioned at a distal end portion of the balloon catheter. A fluid reservoir may be in fluid communication with a lumen, the lumen being in fluid communication with an interior volume of the balloon. A motor may be operably coupled to the fluid reservoir. An actuator may be provided on the handle, the actuator being operably coupled to the motor so that actuation of the actuator in a first direction causes the motor to push fluid from the fluid reservoir through the lumen toward the interior volume of the balloon, and actuation of the actuator in a second direction opposite the first direction causes the motor to withdraw fluid from the interior volume of the balloon toward the fluid reservoir.

According to another embodiment of the disclosure, a method of de-airing a balloon of a balloon catheter may include (i) withdrawing a plunger of a purge syringe while the purge syringe is in fluid communication with the balloon of the balloon catheter and while the purge syringe is not in fluid communication with a fill syringe containing fluid, and monitoring vacuum pressure as a function of plunger displacement, as the plunger of the purge syringe is withdrawn, to create a pressure-displacement line having a slope. The method may also include (ii) advancing a plunger of the fill syringe while the fill syringe is in fluid communication with the balloon of the balloon catheter and while the fill syringe is not in fluid communication with the purge syringe. The method may further include (iii) advancing the plunger of the fill syringe while the fill syringe is in fluid communication with the purge syringe and while the fill syringe is not in fluid communication with the balloon catheter to push remaining air through a one-way valve in fluid communication with the purge syringe. Steps (i) through (iii) may be repeated until the slope of the pressure-displacement line exceeds a threshold slope of a reference pressure-displacement line.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 2A is a highly schematic drawing of a balloon inflation system according to one aspect of the disclosure.

FIG. 2B is a highly schematic drawing of a rapid fluid withdrawal mechanism of the balloon inflation system of FIG. 2A.

FIGS. 3A-B are top and side views, respectively, of a balloon inflation system according to another aspect of the disclosure.

FIG. 3C is a perspective view of a handle of a delivery device coupled to the balloon inflation system of FIGS. 3A-B.

FIGS. 3D-E are views of the handle of the delivery device of FIG. 3C.

FIG. 3F is a side view of a prosthetic heart valve crimped over a deflated balloon of the delivery device of FIG. 3C.

FIG. 3G is an illustration of the balloon of FIG. 3E in an inflated condition.

FIG. 3H is an illustration of the balloon of FIG. 3G with an exemplary pressure sensor used with the balloon.

FIG. 3I is an illustration of the balloon of FIG. 3G with an exemplary strain gauge used on the balloon.

FIG. 4 is a balloon compliance curve illustrating pressure versus volume of the balloon of FIGS. 3F-G.

FIG. 5 is a highly schematic illustration of the interaction between different elements of involved with a prosthetic heart valve implantation using devices described herein.

FIG. 6A is an exemplary display screen during a planning step of a prosthetic heart valve implantation.

FIG. 6B is an exemplary display screen during a mid-procedural step of a prosthetic heart valve implantation.

FIG. 7 is a highly schematic illustration of a de-airing system according to an aspect of the disclosure.

FIG. 8 is a graph illustrating vacuum pressure versus syringe displacement slopes during a de-airing procedure.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

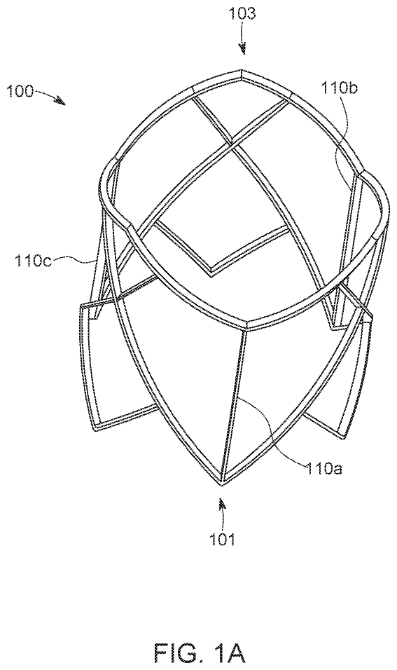

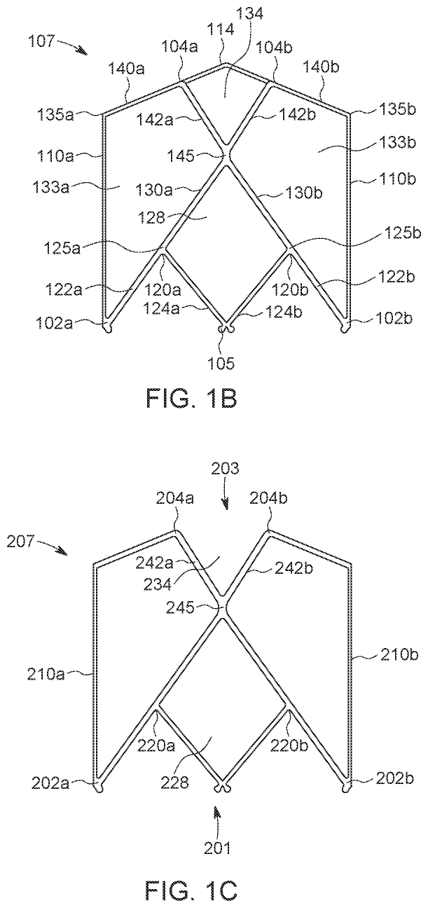

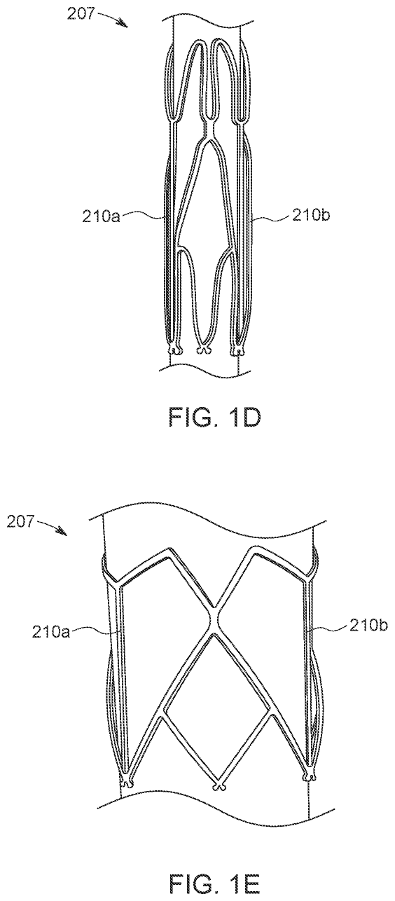

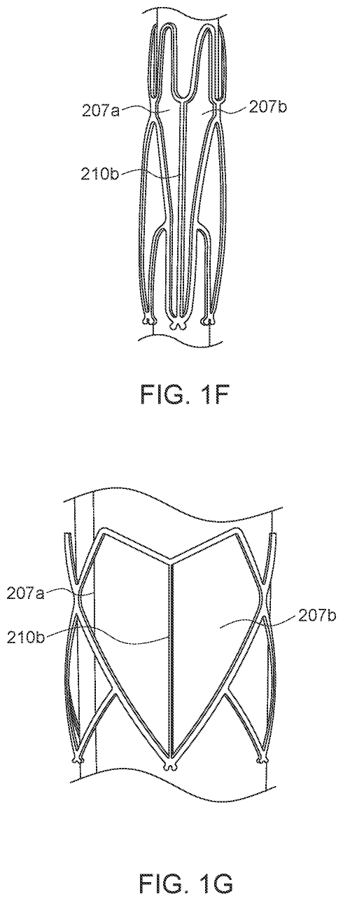

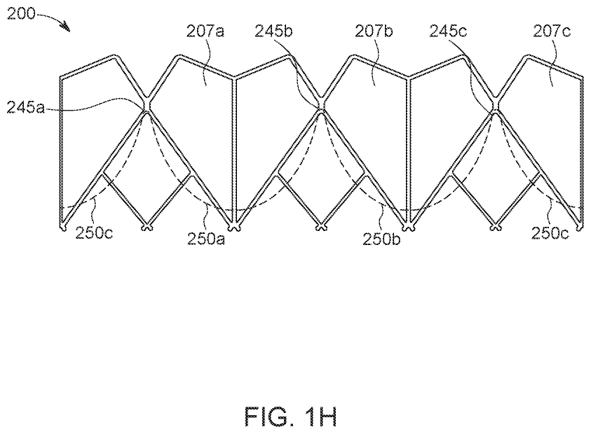

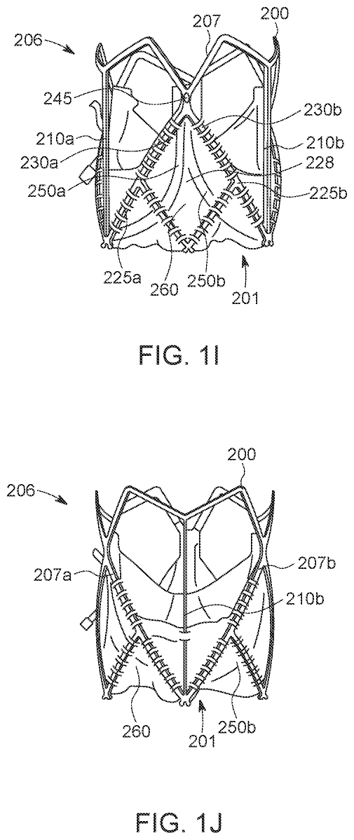

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 250 may include an inflow end disposed substantially along inflow end 201 of stent 200. FIG. 11 shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

Although certain embodiments of balloon-expandable prosthetic heart valves are provided above, it should be understood that the systems, devices, and methods described below for use in inflating, deflating, and/or de-airing a balloon of a balloon catheter may be used with other types of balloon expandable prosthetic heart valves differing from the embodiments described above.

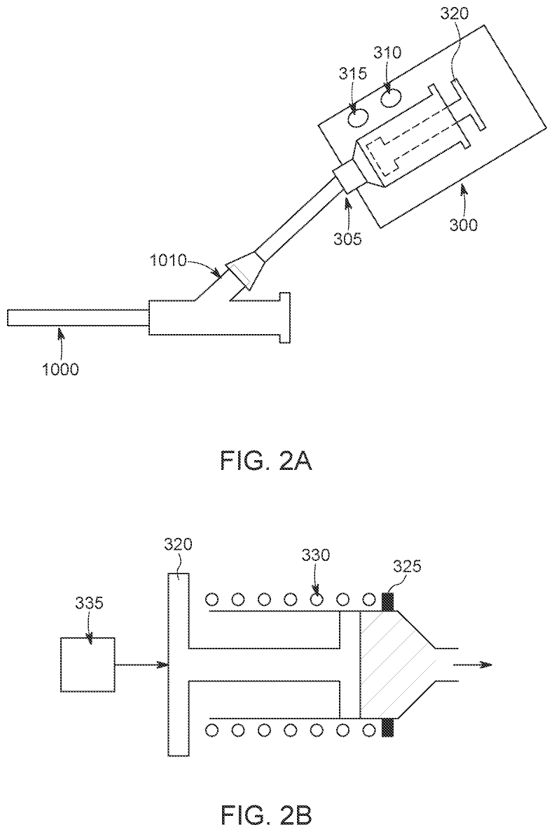

Referring now to FIG. 2A, one embodiment of an automated balloon inflation device 300 is illustrated. In the illustrated embodiment, balloon inflation device may include a housing that securely receives a syringe 305 therein. The housing may secure the body of the syringe 305 in a fixed relation to the housing, with the plunger handle of the syringe 305 received within a moving member that can be driven axially relative to the housing to advance or retract the plunger into the body of the syringe 305. The moving member may be operably coupled to a carriage within the housing, and the carriage may include internal threading that engages external threading of a screw mechanism within the housing, the screw mechanism being operably coupled to a motor within the housing. The motor may be operably coupled to a power supply (e.g. a replaceable or permanent battery within the housing, or to AC mains). With this embodiment, the motor may be activated to rotate the screw mechanism, which in turn may advance or retract (depending on the direction of rotation of the screw mechanism) the moving member, and thus the plunger handle, to drive fluid out of, or into, the syringe. For example, the housing of the balloon inflation device 300 may include a deflation button 310, and an inflation button 315. These buttons 310, 315 may be configured so that holding the corresponding button down activates the motor to turn the screw mechanism in the corresponding rotational direction, which causes the plunger handle to advance or retract at a steady rate of speed (and thus move volume into or out of the syringe at a generally steady volumetric rate), and as soon as pressure on the button is released, the movement stops. In other embodiments, pressing the deflation or inflation button 310, 315 a single time may activate the motor, and pressing the button again may deactivate the motor. In some embodiments, a third button may be provided that may be activated to stop the motor. It should be understood that, during inflation or deflation, the syringe 305 may be coupled to a balloon inflation port 1010 of a balloon catheter 1000, with a lumen extending from the balloon inflation port 1010, through the delivery device containing the prosthetic heart valve in a crimped condition, and into the balloon over which the prosthetic heart valve is crimped. One of the benefits of using balloon inflation device 300, compared to a manually activated syringe, is that the inflation/deflation rate is consistent and allows for controlled inflation and deflation. However, balloon inflation device 300 still requires the user to be fully responsible for determining the proper amount of volume that should be pushed to the balloon to expand the prosthetic heart valve.

Balloon inflation device 300 may be modified to provide additional automation. For example, in another embodiment, balloon inflation device may include an interface that allows the user to input a target volume to which the balloon of the balloon catheter 1000 should be inflated. Upon the user setting the target inflation volume and coupling the syringe 305 to the housing of the balloon inflation device 300, the user may only need to press a single button to begin the process of inflation. The mechanism by which the screw mechanism advances the moving member to depress the handle of the syringe may be identical to that described above, with the inflation occurring at the steady rate for the amount of time required to reach the target inflation volume. The balloon inflation device 300 may be programmed so that, once the target inflation volume is reached, the balloon inflation device 300 holds steady for a predetermined amount of time, and then automatically begins to move the plunger handle of the syringe 305 in the opposite direction to deflate the balloon. In such a system, manual override buttons may be available to stop the process at any desired point. In a similar embodiment, instead of using an off-the-shelf syringe in an external housing, the balloon inflation device 300 may be internalized into a delivery device that has a fluid reservoir instead of a syringe. If such a system were pre-packaged with the prosthetic heart valve, the target volume could be set prior to packaging the system, so that no user input would be required, and a button or other actuator on the delivery device could be pressed to automate the entire process described above in this paragraph. One of the benefits of these two alternative embodiments is that, in addition to eliminating the need for manual balloon inflation and deflation, the target volume will be reached in a predictable and controlled manner. In other words, the system automatically stops inflation once the target volume is reached, effectively eliminating the possibility of inflating the balloon beyond the target volume, which could cause a balloon rupture and/or damage the patient's tissue.

In some embodiments, once the balloon has been inflated and the prosthetic heart valve expanded into place within the native valve annulus, it may be desirable to deflate the balloon very rapidly to minimize the amount of time in which the inflated balloon is filling the valve annulus and blocking blood flow. It should be noted that, during transcatheter prosthetic heart valve deliveries, the heart may be rapidly paced while the prosthetic valve is being deployed. Thus, it may also or alternatively be desirable to deflate the balloon rapidly, after the prosthetic heart valve is deployed, to minimize the amount of time that the heart is rapidly paced. For example, balloon inflation device 300 (or a similar version thereof) may include a biasing member, such as a compression spring 330, having a first end abutting the plunger handle 320 of the syringe 305, and a second end abutting a platform 325 that may be on the housing or coupled to the syringe 305. The motor drive mechanism, illustrated generally as mechanism 335 in FIG. 2B, may work in a similar fashion as described above to drive the plunger handle 320 distally to inflate the balloon. However, as the plunger handle 320 advances distally, the spring 330 begins to compress, applying a proximal force to the plunger handle 320. The proximal force on plunger handle 320 is not strong enough to overcome the motor drive mechanism 335 during inflation. However, once the balloon is inflated and the prosthetic heart valve is deployed, the motor drive mechanism 335 may be decoupled from the plunger handle 320, allowing the spring 330 to rapidly decompress, forcing the plunger handle 320 proximally and causing rapid withdrawal of fluid from the balloon back into the body of the syringe 305. Any suitable mechanism may be provided to decouple the motor drive mechanism 335 from the plunger handle 320. For example, if the plunger handle 320 is received within (or coupled to) a moving member that is operably coupled to a carriage that is engaged with a threaded screw mechanism, a coupling between the moving member and the carriage, or between the carriage and the threaded screw, may be disconnected to allow the compression spring 330 to decompress. In other embodiments, a split nut may be used such that the nut is formed as two or more pieces that can come together to engage the threads or separate to disengage the threads to allow for rapid decoupling of the motor drive mechanism 335 from the plunger handle 320. In still other embodiments, a single-sided thread tooth may be provided that is allowed to translate or “rock” into place to engage or disengage the threads of the threaded screw mechanism. However, it should be understood that the compression spring 330 is optional. For example, the speed at which the plunger handle 320 is driven by motor drive mechanism 335 may be fast enough that, upon driving the plunger handle 320 proximally to deflate the balloon, the speed with which the balloon deflates is sufficient to avoid any significant negative patient outcomes of duration of blood flow blockage via the balloon (and/or duration of rapid pacing of the heart).

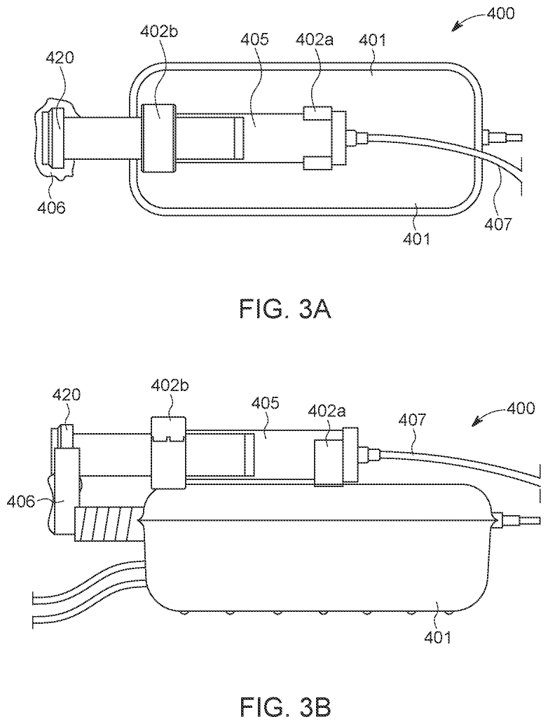

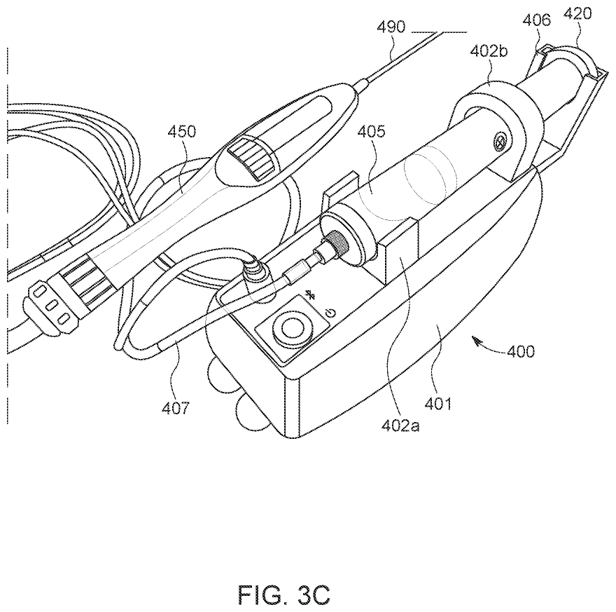

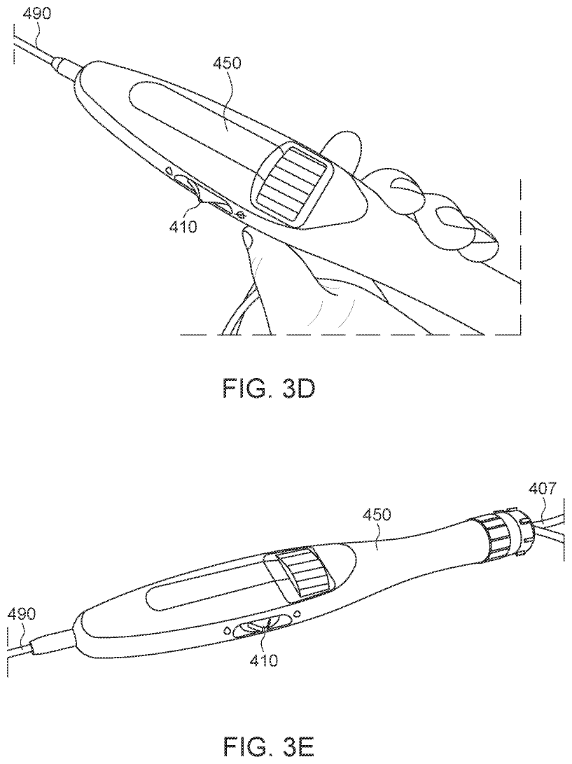

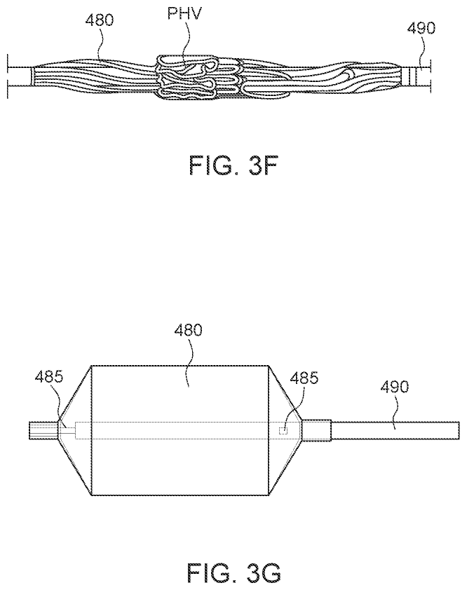

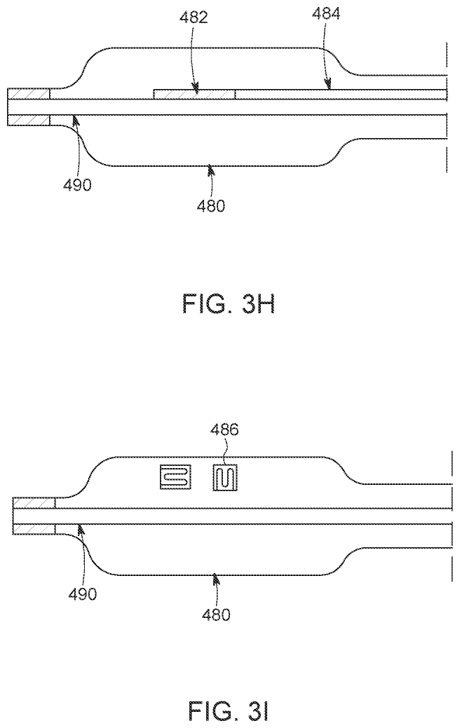

FIGS. 3A-B illustrate another embodiment of a balloon inflation system 400. Similar to balloon inflation device 300, balloon inflation system 400 may include a housing 401 that houses one or more components, which may include a motor, one or more batteries, electronics for control and/or communication with other components, etc. Housing 401 may include one or more fixed cradles to receive syringe 405. In the illustrated embodiment, a distal cradle 402a is provide with an open “C”- or “U”-shaped configuration so that the distal end of the syringe 405 may be snapped into or out of the distal cradle 402a. A proximal cradle 402b may also be provided, which may have a “C”- or “U”-shaped bottom portion hingedly connected to a “C”- or “U”-shaped top portion. This configuration may allow for the proximal end of the outer body of the syringe 405 to be snapped into the bottom portion of cradle 402b, and the top portion of cradle 402b may be closed and connected to the bottom portion to fully circumscribe the outer body of the syringe 405 to lock the syringe 405 to the housing 401. It should be understood that more or fewer cradles, of similar or different designs, may be included with housing 401 to help secure the syringe 405 to the housing in any suitable fashion.

The balloon inflation system 400 may include a moving member 406. In the illustrated embodiment, moving member 406 includes a “C”- or “U”-shaped cradle to receive the plunger handle 420 therein, the cradle being attached to a carriage that extends at least partially into the housing 401. The carriage of the moving member 406 may be generally cylindrical, and may include internal threading that mates with external threading of a screw mechanism (not shown) within the housing that is operably coupled to a motor. In some embodiments, the carriage may have the general shape of a “U”-beam with the flat face oriented toward the top. The moving member 406 may be rotationally fixed to the housing 401 via any desirable mechanism, so that upon rotation of the screw mechanism by the motor, the moving member 406 advances farther into the housing 401, or retracts farther away from the housing 401, depending on the direction of rotation of the screw mechanism. While the plunger handle 420 is coupled to the moving member 406, advancement of the moving member 406 forces fluid from the syringe 405 toward the balloon, while retraction of the moving member 406 withdraws fluid from the balloon toward the syringe. It should be understood that the motor, or other driving mechanism, may be located in or outside the housing 401, and any other suitable mechanism may be used to operably couple the motor or other driving mechanism to the moving member 406 to allow for axial driving of the plunger handle 420.

As shown in FIGS. 3A-C, the distal end of syringe 405 may be coupled to tubing 407 that is in fluid communication with a lumen (e.g. of a balloon catheter) that leads to the balloon 480 at or near the distal end of the delivery device. Tubing 407 may allow for the passage of the fluid (e.g. saline) from the syringe 405 toward the balloon 480, for example via one or more fluid ports 485 (shown in FIG. 3F) in a shaft of the balloon catheter 490, and vice versa.

Although not separately numbered in FIGS. 3A-B, the housing 401 may include one or more cables extending from the housing, for example to allow for transmission of power (e.g. from AC mains or another component with which the cable is coupled) and/or transmission of data, information, control commands, etc. For example, as described in greater detail below, one cable may couple the housing 401 to a handle 450 of a delivery device so that controls on the handle 450 may be used to activate the balloon inflation system 400 in the desired fashion. Another cable, as described in greater detail below, may couple to a computer display or similar device to provide information regarding the inflation of the balloon 480. However, it should be understood that any transmission of data or information may be provided wirelessly instead of via a wired connection, for example via a Bluetooth or other suitable connection.

Referring now to FIGS. 3D-E, the handle 450 of the delivery device may include one or more knobs or actuators to manipulate different features of the delivery device. For example, a rotating knob near a center of the handle 450 may provide for catheter deflection, while other knobs or actuators (e.g. near the proximal end of the handle 450) may allow for rotational and/or axial adjustment of the balloon 480 (and the prosthetic heart valve PHV mounted thereon) to help get a precise alignment between the native valve annulus and the prosthetic heart valve PHV before deployment. It should also be understood that, although a single shaft is shown and labelled as balloon catheter 490, the delivery device may include more than one coaxial catheter shaft to provide for desired functionality, including relative axial movement between catheter shafts in the stack, etc.

Referring still to FIGS. 3D-E, the handle may include an actuator 410 for controlling inflation and deflation of the balloon 480 via the control of the balloon inflation system 400. For example, the actuator 410 may be in the form of a sliding button that has a neutral center position, and which may be advanced distally to inflate the balloon 480, and pulled proximally to deflate the balloon 480. In one example, the actuator 410 remains in the distal or proximal position after being moved from the neutral position. In another embodiment, the actuator 410 may be biased to the neutral position so that, as soon as a user releases force being applied to the actuator 410, the actuator 410 automatically moves back to the neutral position in which fluid is being neither passed into nor withdrawn from the balloon 480. As should be understood, upon sliding the actuator 410 forwards or backwards, control signals may be sent (for example via a cable) from the handle 450 to the balloon inflation system 400, causing the motor to drive the moving member 406 forward or backward to push fluid toward, or withdraw fluid from, the balloon 480. Although one example of a sliding actuator 410 is shown in FIGS. 3D-E, it should be understood that other actuators, such as individual buttons, a rotating knob, etc. may be provided on handle 450 to allow for control of the balloon inflation system 400.

It should also be understood that actuator 410 may provide for a binary control style, in which inflation (or deflation) is either occurring at a set rate, or not occurring at all. In other embodiments, the actuator 410 may have variable inflation speed control. For example, the user may be able to press the actuator 410 distally (or proximally), and the farther the user presses the actuator distally (or proximally), the faster the balloon inflation system 400 inflates (or deflates) the balloon 480. This variable volume flow rate may be desirable to allow the user even finer control of the inflation or deflation of the balloon 480, which may be particularly useful when the balloon is close to reaching the target size/volume.

Referring to FIG. 3F, an example of a prosthetic heart valve PHV, which may include a stent similar to stent 100, is shown crimped over the balloon 480 of the balloon catheter 490 while the balloon 480 is in a deflated condition. Upon sliding actuator 410 distally, the balloon inflation system 400 causes fluid to be pumped from the syringe 405 into the balloon 480, for example through a lumen within balloon catheter 490 and into one or more ports 485 located internal to the balloon 480. In the particular illustrated example of FIG. 3G, which omits the prosthetic heart valve PHV from the figure, a first port 485 may be one or more apertures in a side wall of the balloon catheter 490, and a second port 485 may be the distal open end of the balloon catheter 490, which may terminate within the interior space of the balloon 480.

The balloon inflation system 400 may be used in a semi-manual method in which pressure or other sensors do not provide real-time feedback for the user. In such an example, the distal end of the delivery device may be delivered into the patient while the prosthetic heart valve PHV is crimped on the deflated balloon 480. For example, the distal end of the delivery device may be advanced through a patient's femoral artery, and eventually turning to traverse the aortic arch, with or without the help of a steering mechanism (e.g. pull wires coupling the handle 450 to a distal end portion of the balloon catheter 490. Once the prosthetic heart valve PHV is positioned within the native aortic valve annulus, the balloon 480 may be inflated and the prosthetic heart valve PHV expanded into the native aortic valve annulus. This deployment may be performed while the heart of the patient is being rapidly paced. To inflate the balloon 480, the user may slide the actuator 410 distally, which sends a signal to the balloon inflation system 400 to start driving the moving member 406 distally, which causes the plunger handle 420 to depress, forcing fluid such as saline within the syringe 405 to pass through tubing 407, through the balloon catheter 409, and into the balloon 480, causing the balloon to expand and force the prosthetic heart valve PHV to expand into the native aortic valve annulus. The user may visualize the process, for example under fluoroscopy, and when the user confirms that the prosthetic heart valve PHV is at the desired size and position, the actuator 410 may be slid proximally to reverse the directional movement of the moving member 406 to withdraw fluid from the balloon 480 back into the syringe 405, causing the balloon 480 to deflate. With the balloon 480 deflated, and the prosthetic heart valve PHV expanded into place, the rapid pacing of the heart may be stopped and the heart may begin beating normally again, with the prosthetic heart valve PHV now regulating blood flow between the left ventricle and the aorta. In some embodiments, the user may enter a target or maximum inflation volume, either directly into the balloon inflation system 400 (for example via a user interface provided thereon) or into a computer operatively coupled to the balloon inflation system 400. If such a target or maximum inflation volume is used, the motor in the balloon inflation system may be programmed to turn off once the balloon inflation system 400 has pushed the target inflation volume out of the syringe 405. In some embodiments, the target or maximum inflation volume may be manually overridden, for example if the user determines that additional balloon expansion is required to properly expand the prosthetic heart valve PHV into the native aortic valve annulus. It should be understood that the volume of fluid moving through the balloon inflation system 400 may be tracked by any suitable method. In one embodiment, the inner diameter of the syringe 405 is known, and the travel distance of the moving member 406 is known, so the resulting volume exiting (or entering) the syringe 405 may be determined from a simple calculation. In other embodiments, fluid sensors may be incorporated into the system to assist with tracking total fluid volume moved into or out of the syringe 405 in real time.

Although the above-described use of balloon inflation system 400 may provide significant benefits, including control and precision, compared to prior known fully manual inflations of balloons to implant a prosthetic heart valve, additional data may be used with balloon inflation system 400 to provide additional useful features. For example, pressure within the balloon catheter line may be actively monitored to provide additional data that may be used during the deployment of the prosthetic heart valve PHV. In order to obtain pressure data inside the fluid line of the balloon catheter, one or more pressure sensors may be provided within fluid path. Such locations may include, for example, within the balloon catheter lumen at a location inside handle 450, within or adjacent to the syringe 405 (e.g. near the tip), or inside the a balloon 480 (e.g. directly on an interior surface of the balloon or on the inside or outside of the portion of the shaft of the balloon catheter 490 which the balloon 480 surrounds. In some embodiments, a pressure wire may be provided within the balloon catheter, for example with the sensor housing positioned within the balloon 480. FIG. 3H shows one embodiment in which a pressure sensor 482 is mounted to a portion of a shaft of the balloon catheter 490 within the balloon 480, with a wire connection 484, such as an electrical or optical wire connection, connecting the pressure sensor 482 to another component to read and/or transmit the sensor information. The pressure sensor 482 may be a MEMS based sensor, although other types of sensors may be suitable. Placing the pressure sensor 482 within the balloon 480 may allow for a direct measure of the pressure internal to the balloon, which may avoid potential errors if the pressure measurement is taken farther away from the balloon 480, such as the inflation port or at the syringe 405. However, it should be understood that the use of a pressure sensor that is not directly within the balloon 480 may nonetheless provide satisfactory measurements, and the particular sensing mechanism by which real-time pressure within the balloon catheter is measured may take any other suitable form.

It may also be desirable to determine the area and/or diameter of the balloon 480 during inflation. One way in which the area and/or diameter of the balloon 480 may be determined is based on previously determined relations between pressure or volume and diameter. For example, for a balloon 480 having a particular construction, correlations may be determined that are generally applicable so that, based on the calculated volume that has been passed toward the balloon 480, the area and/or diameter of the balloon 480 may be estimated based on the known correlation between pressure or volume and size. Thus, if a final desired size is known for the prosthetic heart valve PHV, an algorithm based on that correlation may be applied to determine the total volume that should be pushed to the balloon 480 (or the pressure needed to reach the desired size). In other embodiment, sensors may be provided on the balloon 480 to provide information regarding the diameter of the balloon 480. For example, as shown in FIG. 3I, one or more strain gauges 486 may be mounted or otherwise affixed to the outer wall of the balloon 480 to provide direct measures of the diameter of the balloon 480. The strain gauges 486 may be mounted radially in order to measure hoop strain, or axially to determine diameter. Generally, it may be more desirable for radial mounting compared to axial mounting if the desire is to measure the diameter. The general formula for radial strain in a cylindrical pressure vessel is that radial strain is equal to the product of the pressure and radius, divided by the product of the modulus and wall thickness. Axial strain is half of radial strain, so it can be used as a less direct measure of radial strain, which may be less preferred.

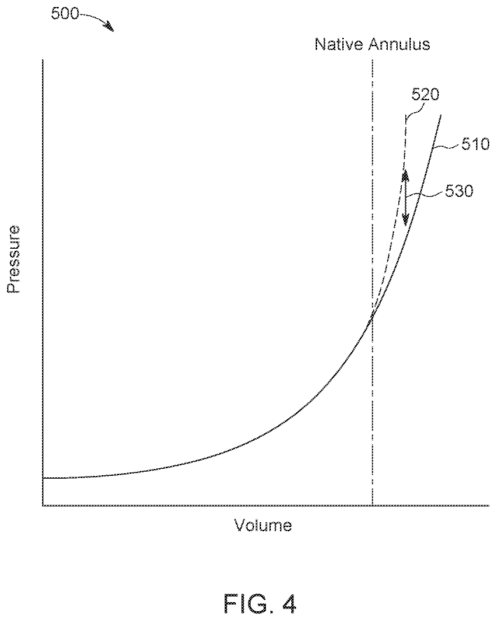

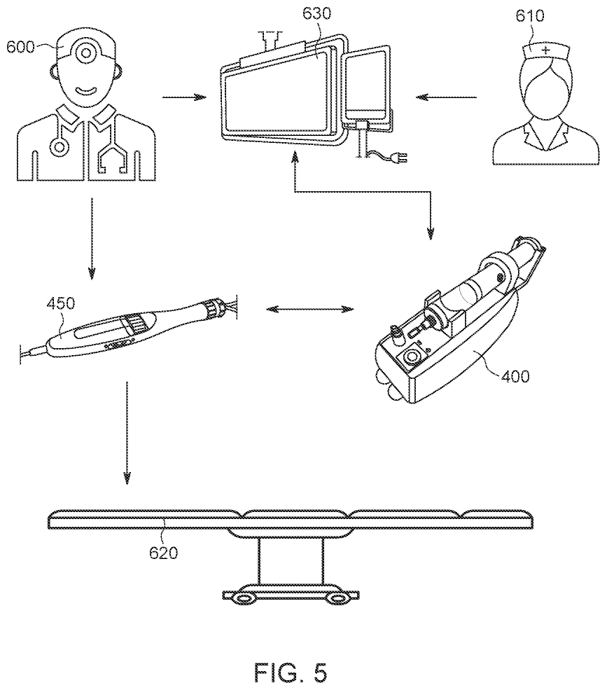

FIG. 4 illustrates a balloon compliance curve for the balloon 480 illustrating the relationship between balloon pressure and balloon volume. The solid line represents the baseline or “open air” pressure-volume curve 510 when the balloon 480 is inflated without coming into contact with other structures. However, once the balloon 480 makes contact with a surface, such as the aortic valve annulus, the pressure-volume curve shifts from the baseline, shown in the dashed line 520. Curves 510, 520 are identical prior to the balloon 480 inflating into contact with the native aortic annulus. However, upon contact, the pressure-volume curve shifts from baseline 510 by an amount, with this change or delta represented by arrow 530. This delta 530 is the result of the native tissue compliance applying a compressive force against the expanded balloon. This information may be utilized to help determine when the prosthetic heart valve PHV has been expanded the desired amount. For example, a particular amount of deviation 530 between the baseline pressure-volume curve 510 and the actual pressure-volume curve 520 during implantation may be determined as the amount of deviation 530 that corresponds to optimal prosthetic heart valve PHV expansion within the aortic valve annulus. This value of desired deviation 530 may be determined, for example, via testing across multiple patients or by analysis of data of a number of actual implantations.

As noted above, the volume of fluid passing from syringe 405 into balloon 480 may be tracked in real time, and the pressure may also be tracked in real time via one or more of the pressure sensors described above. Thus, during a valve implantation using prosthetic heart valve PHV and balloon inflation system 400, the real time pressure-volume curve 520 may be displayed, along with the expected baseline press-volume curve 510, for reference by the user, allowing the user to use the displayed data to confirm the desirability of the procedure or otherwise to alter the procedure based on the data.

For example, FIG. 5 is a schematic representation of a procedural set-up that includes a first user, such as a physician 600, a second user, such as a support personnel 610, a procedural table 620 for the patient, one or more computers and/or displays 630, the balloon inflation system 400 and the delivery device, including handle 450. Some of the various interactions within the set-up are briefly described below before a further detailed description of specific examples of intraoperative uses of data obtained during the procedure. As described above and reiterated here, fluid may pass in either direction between the balloon inflation system 400 and the handle 450 of the delivery system, and the instruction signals for activating the balloon inflation system 400 may pass from the handle 450 to the balloon inflation system 400. The physician 600 may manually control the delivery system, including using handle 450 to implant the prosthetic heart valve PHV into the patient on the table 620, including via controlling the inflation and deflation of the balloon 450 via the actuator 410. During the procedure, data obtained from the procedure, such as volume that has passed from the balloon inflation system 400 toward the balloon 480 (or vice versa), the current area of the balloon 480 (which may be calculated based on the fluid volume), and the current pressure within the system, may all be transmitted to and/or displayed on the one or more computers and/or displays 630. As noted above, the data transmission may be via a wired or wireless connection. The physician 600 may view the display(s) 630, which may include the above-noted data, as well as other information such as fluoroscopic images of the patient's anatomy. The support personnel 610 may similarly view the data on the display(s) 630, and either the physician 600 or the support personnel 610 may input parameters, such as a target volume for the inflation of balloon 680 via the computer(s) and/or display(s) 630. The computer(s) and/or display(s) 630, in turn, may communicate such parameters to the balloon inflation system 400, for example by setting the target volume such that the balloon inflation system 400 does not inflate the balloon 480 beyond the target volume, unless either user 600, 610 overrides the balloon inflation system 400.

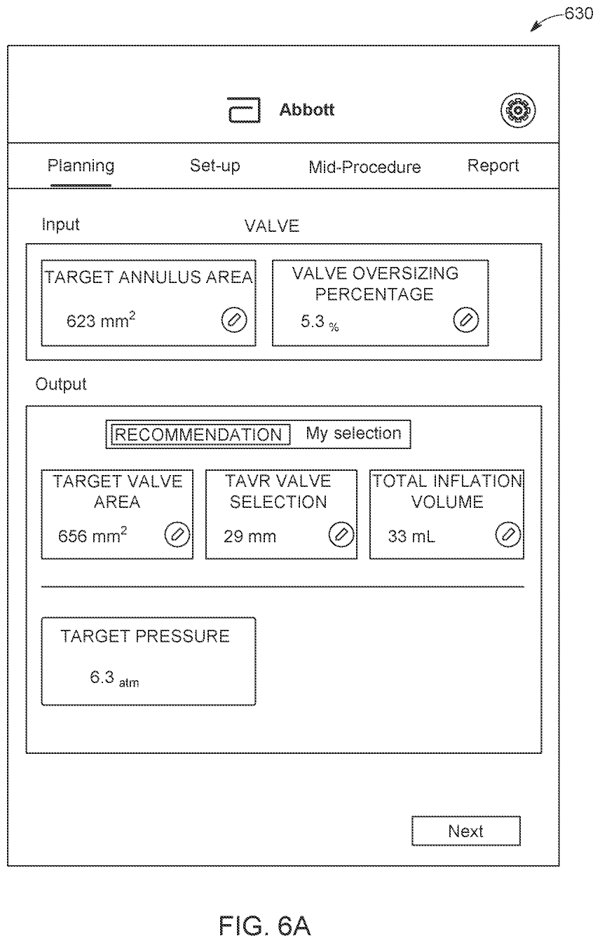

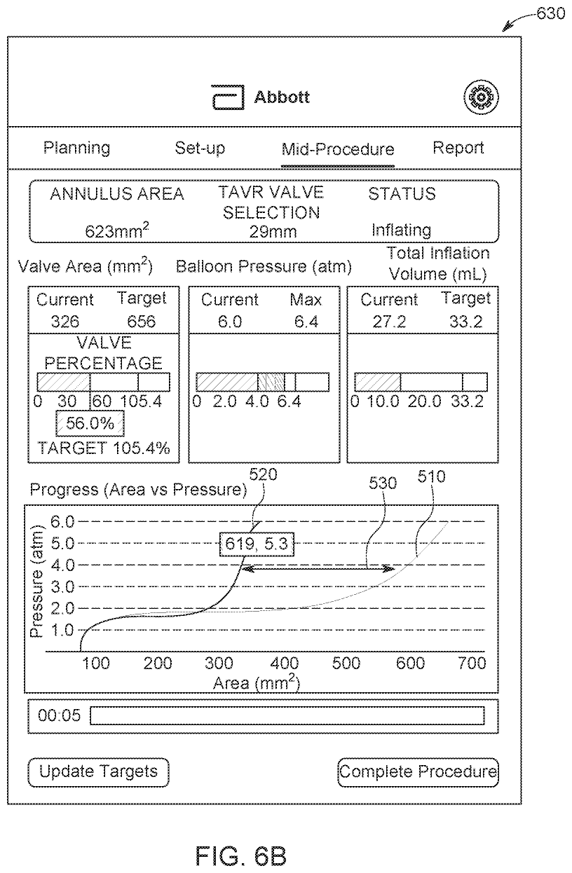

FIG. 6A illustrates an exemplary screen that may be shown on the computers and/or displays 630 as part of a planning stage prior to implanting the prosthetic heart valve PHV into the patient. In this exemplary screen, one or more inputs may be entered for use during the procedure. One exemplary input is the target annulus area, which represents the size of the patient's native valve annulus. In this particular example, the value entered is 623 mm². Another exemplary input is the desired oversizing percentage of the prosthetic heart valve PHV. In other words, it is often desirable to target an area/size for the prosthetic heart valve PHV that is larger than the area/size of the patient's native valve annulus, for example to create enough friction to help maintain the prosthetic heart valve PHV in place during normal operation of the prosthetic heart valve PHV. In this particular example, the value entered for the percent oversizing is 5.3%. It should be understood that all numbers and values provided with respect to FIGS. 6A and 6B are merely exemplary and are not intended to be limiting, but rather illustrate one example of inputs and outputs to better illustrate the related concepts. Based on the input during the planning stages, certain outputs may be provided for review and confirmation by the physician 600 and/or support personnel 610, such as the target area for the prosthetic heart valve PHV, which may be calculated by applying the oversizing percentage to the patient's annulus area. A particular size prosthetic heart valve PHV may be suggested by the output as well. For example, prosthetic heart valves PHV are typically provided in a different selection of sizes, and the physician 600 will choose the appropriate size selection for the particular patient. In this example, a prosthetic heart valve PHV size of 29 mm is recommended based on the inputs. Another output that may be provided to the users 600, 610 is a suggested total inflation volume that should be pushed from the balloon inflation system 400 to achieve the desired expansion size of the prosthetic heart valve PHV. In this particular example, a total inflation volume of 33 mL is suggested. The suggested inflation volume may be provided base on, for example, pre-determined correlations derived from testing that relate inflation volume to valve area upon expansion of the balloon 480. Still another output that may be provided is a target pressure for the balloon 480 to achieve the desired expansion size of the prosthetic heart valve PHV. In this particular example, a target pressure is provided ad 6.3 atm. It should be understood that, although various recommended values are output based on the input data, the physician 600 has the control to override the recommended values based on his or her experience.

FIG. 6B illustrates an exemplary screen that may be shown on the computers and/or displays 630 as part of the mid-procedure stage of implanting the prosthetic heart valve PHV into the patient following the planning stage shown in FIG. 6A. The patient's annulus area and selected size of the prosthetic heart valve PHV from the planning stage of FIG. 6A may be displayed on the mid-procedure screen of FIG. 6B, as well as a current status of the procedure, for example “inflating” or “deflating.” The mid-procedure screen of FIG. 6B may show a plurality of sections that provide a current procedure parameter versus the target procedure parameter in order to assist the users 600, 610 in understanding the progress of the deployment of the prosthetic heart valve PHV. For example, a valve area section may provide the target prosthetic heart valve PHV expansion size/area compared to the current prosthetic heart valve PHV expansion size/area during inflation of the balloon 680. The illustrated screen 630 shows the previously chosen target size of 656 mm² compared to the current mid-inflation size of the prosthetic heart valve PHV of 326 mm². In addition to providing the values, a graph, such as a progress bar, may be shown illustrating the current expansion size as a percent of the patient's valve annulus size compared to the desired expansion size as a percent of the patient's valve annulus size. The target value from the planning stage, which in this example is a 5.4% oversizing, is indicated on the progress bar (e.g. 105.4% of the patient's annulus size), along with the current status (e.g. a mid-procedure size of the prosthetic heart valve PHV of 56.0% of the patient's annulus size). Similar information panels with progress bars may be provided for other parameters, such as a current balloon pressure (e.g. of 6.0 atm) versus the planned target balloon pressure (e.g. of 6.4 atm). Another information panel for current inflation volume (e.g. 27.2 mL at the illustrated stage of the procedure) versus the target inflation volume (e.g. 33.2 mL from the planning stage) may be provided along with a graphical representation, for example via a progress bar that includes an indicator of the target inflation volume.

FIG. 6B also illustrates a graph that plots the pressure within the balloon 480 versus the area of the balloon 480 as the procedure continues. Similar to the compliance curve in FIG. 4, a baseline pressure-area curve 510 may be provided as a static, known relation that would be expected for inflating the balloon 480 in “open air.” As the balloon 480 inflates, the actual procedural pressure-area curve 520 may be plotted as the pressure is detected while the area is either sensed (e.g. using strain gauges 486) or computed (e.g. based on known correlations between fluid volume and area for the balloon 480. As shown in FIG. 6B, a deviation 530 between the procedural pressure-area curve 520 from the baseline pressure-area curve 610 results. It should be understood that the large deviation 530 shown in FIG. 6B is merely for illustrative purposes, and the deviation 530 of the size shown is not intended to represent an actual expected level of deviation. Regardless, the deviation 530 between the baseline curve 510 and the procedural curve 520 may provide important information to the users 600, 610 that may be utilized to confirm that the procedure is proceeding as intended, or otherwise that a potential problem has occurred that needs to be investigated and/or addressed. For example, as noted above, a known target deviation 530 between the baseline curve 510 and the mid-procedure curve 520 may be either set or understood to be a deviation that is desired. If the illustrated deviation 530 is near or equal to the target deviation 530, and the other target parameters (e.g. prosthetic heart valve PHV size, balloon pressure, and/or inflation volume) are all at or near their target values, the physician 600 may determine that the procedure has met all of the targets and that the prosthetic heart valve PHV has been appropriately deployed. It should be understood that the physician 600 may use other information, including fluoroscopic images and his or her general experience and knowledge, to aid in this determination. However, if the procedural pressure-area curve 520 is deviating from the baseline curve 510 significantly more than expected, such a deviation 530 may be indicative of a potential problem. For example, in the illustrated example of FIG. 6B, the pressure of the balloon 480 has increased much sooner than expected, meaning that despite the prosthetic heart valve PHV having been only partially expanded toward the target size, the balloon 480 and prosthetic heart valve PHV are experiencing significantly higher forces than expected, which may be the result of the native tissue pressing against the prosthetic heart valve PHV. Reasons for this deviation may include, for example, an incorrectly measured size of the patients' valve annulus and/or significantly greater calcification of the native valve than expected. If the physician 600 continued inflating the balloon 480 despite the large deviation 530 shown, the annulus and/or balloon 480 may be at risk of rupture. Thus, the physician 600 may pause the inflation of the balloon 480 using the actuator 410 on the handle 450, and assess the situation to determine the cause of the deviation 530, and may adjust the procedure accordingly. In some embodiments, the physician 600 or support personnel 610 may interact with the computer(s) and/or display(s) 630 to update the target parameters based on information learned from the assessment following pausing of inflation. In some embodiments, the physician 600 or support personnel 610 may look to the live fluoroscopic images for valve expansion and if it is believed that the prosthetic heart valve is anchored, the balloon may be deflated for further investigation using, for example, fluoroscopy and contrast injections.

In some embodiments, the computer(s) and/or display(s) 630 may be programmed to provide alerts to the users 600, 610 when parameters are approaching, at, and/or exceeding the target parameters. Such alerts may be purely information, or may otherwise cause a procedural change. For example, when the sensed pressure of the balloon 480 achieves the target pressure (or otherwise exceeds the target pressure, or exceeds the target pressure by a pre-determined buffer value, for example 5%), the computer(s) and/or display(s) 630 may create a purely informational alert that the target pressure has been reached or exceeded (either by any amount or the predetermined buffer amount), or may create an alert that also signals the balloon inflation system 400 to stop inflating the balloon 480. If the alert causes such an action, the users 600, 610 will be able to override that alert by dismissing it, for example via interaction with the computer(s) and/or display(s) 630, and then continue inflating the balloon 480 if deemed appropriate to do so. Other similar types of alerts for other target values may be similarly provided.

The information from the procedure may not only be useful for that specific procedure, but the data from numerous procedures may be aggregated and used to inform how to optimize later procedures. For example, a database of information (preferably that anonymizes patient information) may be created that stores information relevant to parameters encountered during a particular procedure, as well as the outcome of the procedure. As one specific example, the effectiveness of inflating the balloon 480 to a particular pressure and particular size in view of a patient's native annulus size may be stored in a database, as well as the resulting effectiveness of the implant. With enough data, desired target parameters for future patients may be determined based on prior procedural results. For example, if a patient of a particular age, sex, and having a particular size native aortic valve annulus is a candidate for a prosthetic heart valve PHV implantation, the patient's information may be compared to other similar patients who have undergone a prosthetic heart valve PHV implantation, and the target parameters for the future patient's procedure may be based, at least in part, on actual parameters found in prior similar patients who have had successful prosthetic heart valve PHV implantations. As more data is accumulated, more accurate and sophisticated algorithms may be created to predict the target procedural parameters for an individual patient to optimize patient outcomes. For example, with enough data from prior procedures, the system may become highly sensitive and able to determine subtle changes in the delivery and/or deployment of the prosthetic heart valve PHV based on recognizing patterns of detected pressure changes. For example, the system may alert the user(s) to initial contact with tissue based on subtle pressure reading changes during deployment of the prosthetic heart valve PHV within the valve annulus. Other situations may provide characteristic pressure readings, such as contact with highly calcified tissue, tearing of any native tissue due to balloon expansion forces, etc. The system may alert the user(s) to each of these scenarios based on the detected characteristic pressure change readings. And it should be understood that these characteristic pressure change readings are not necessarily limited to the deployment phase of the prosthetic heart valve PHV, but may be detected during delivery after the prosthetic heart valve PHV has been introduced into the vasculature. For example, during delivery of the prosthetic heart valve PHV, the system may detect characteristic pressure change readings indicative of certain events, such as the prosthetic heart valve PHV shifting positions relative to the balloon, contact of the prosthetic heart valve PHV with the luminal tissue of the vasculature, etc.

While the balloon inflation systems described above may provide significant utility in inflating the balloon 480 during an implantation of a prosthetic heart valve PHV, the balloon inflation systems may also have utility in preparing the balloon catheter for later use, including for example in de-airing the system. During use of the balloon catheter 490 described above, the interior fluid line within the balloon catheter 490 and extending into syringe 405 is a closed system. It is generally important that the amount of air within that closed system is minimized. Having air within the system may create problems or potential problems. For example, if the balloon 480 were to burst and air were within the system, air could be released into the blood stream with the potential to cause blockages of blood flow, which could result in a stroke or another medical crisis. Further, air within the system may cause pressure or other readings to be less accurate than if there were no (or a minimum acceptable level of) air within the system. Currently, in order to de-air a balloon catheter prior to use, a manual process is performed in which a vacuum in the balloon catheter is created with a first syringe, and then fluid is pushed into the balloon catheter with a separate syringe, and the cycle is repeated to fill the balloon catheter with fluid and purge air remaining in the balloon catheter. However, in order to confirm that there is no (or a minimal acceptable level of) air in the balloon catheter, a visual check is done which may be a relatively subjective analysis with a potentially high risk of error given the human factor involved. The balloon inflation systems described herein may be used to automate the de-airing process while also creating a more objective, data-based confirmation of the amount of air remaining in the balloon catheter following the de-airing procedure.

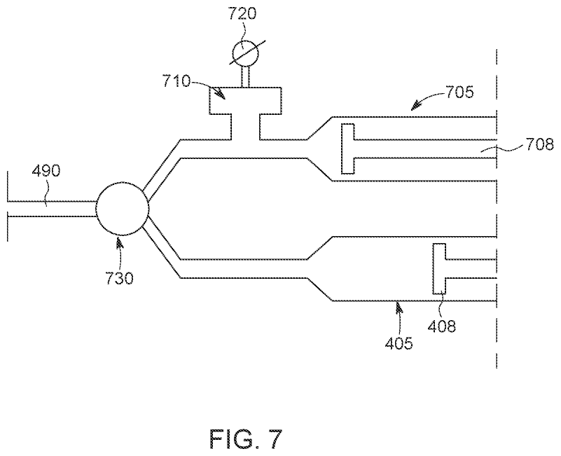

FIG. 7 is a schematic illustration of a de-airing system that includes a fill syringe 405 and a purge syringe 705. Fill syringe 405 and purge syringe 705 may be substantially identical, and each may be part of a balloon inflation system similar to system 400 described above. Each syringe 405, 705 may be fluidly coupled to the balloon catheter 490 via a three-way stopcock valve 730, the stopcock valve 730 allowing for any component of the system being fluidly isolated from the other two components. An air catch 710 may be positioned between the purge syringe 604 and the valve 730, with a one-way check valve 720 being positioned downstream of the air catch 710.

In an exemplary use of the de-airing system of FIG. 7, the balloon catheter 490 is initial devoid of fluid. The inflation syringe 405 is pre-filled with the desired fluid (typically saline) with the inflation syringe plunger 408 positioned proximally, and the plunger 708 of purge syringe plunger 708 is advanced fully distally. The syringes 405, 705 are coupled to the balloon catheter 490 via the three-way valve 730 while in this position. The three-way valve 730 is positioned so that the syringes 405, 705 are in fluid communication with each other, but isolated from the balloon catheter 490. While the three-way valve 730 is in this position, the plunger 408 of the inflation syringe 405 is depressed, pushing fluid toward purge syringe 705. During this step, air within the lines is purged via the air catch 710 and the check valve 720. It should be understood that a relatively small percentage of the fluid within inflation syringe 405 may exit the inflation syringe 405 during this step. Next, the three-way valve 730 is positioned to fluidly connect the purge syringe 705 and the balloon catheter 490, isolating the inflation syringe 405 from the other components. The plunger 708 of the purge syringe 705 is withdrawn to create a vacuum in the balloon catheter 490. During this step, which may be referred to as a first step, the pressure within the balloon catheter 490 may be measured and recorded as a function of the displacement of the purge syringe plunger 708, using any suitable pressure sensor device, including those described above. While the vacuum is maintained, the three-way valve 730 may be positioned to fluidly connect the balloon catheter 490 and the inflation syringe 405, isolating the purge syringe 705. During this step, which may be referred to as a second step, the plunger 408 is advanced distally to push fluid (or to allow fluid to be pulled because of the vacuum) into the balloon catheter 490. Then, the three-way valve is positioned to fluidly connect the syringes 405, 705, isolating the balloon catheter 490. In this step, which may be referred to as a third step, the plunger 408 may be depressed to force air captured in the air catch 710 out of the check valve 720. The process of the steps referred to as the first, second, and third steps may be repeated in sequence, with a pressure vs. plunger 708 displacement being measured and recorded during the first step of each cycle.

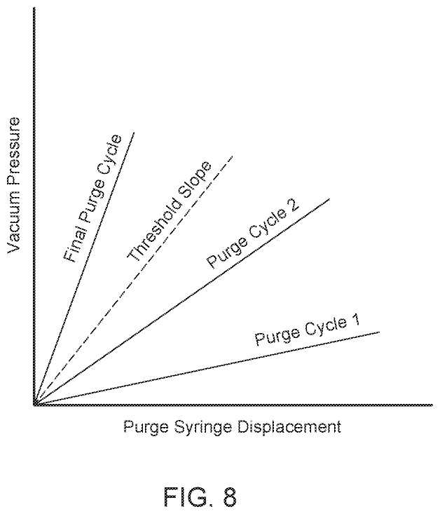

As noted above, during each cycle of the de-airing process, a pressure vs. displacement measurement is recorded as the purge syringe 705 pulls a vacuum while in fluid connection with the balloon catheter 490. When there is air in the balloon catheter 490, the vacuum pressure achieved during this first step is relatively small because the air in the system expands into the purge syringe 705. However, an amount of that air is purged from the system during each cycle of the steps above referred to as first, second, and third steps. Thus, the second time that a vacuum is pulled by the purge syringe 705, while there is less air in the system as the previous cycle, the vacuum pressure achieved is greater than the first cycle. As the cycle is repeated and more air is purged from the system, the vacuum pressure created by a given displacement of the purge syringe plunger 708 increases. As shown in FIG. 8, during each purge cycle, as more air is removed from the system, the slope of the vacuum pressure vs. displacement relationship increases. Because these slopes generally follow mathematical models, a threshold slope may be determined that represents when the amount of air remaining in the balloon catheter 490 is at an acceptable level. Thus, the purge cycle may continue until the slope of the vacuum pressure vs. syringe displacement is greater than or equal to the minimum threshold slope. At this point, the balloon catheter 490 has been purged enough so that no more than an acceptable amount of air remains within the balloon catheter 490. In a similar but alternative embodiment, a minimum vacuum level for a given syringe displacement may also be set as the threshold level which, upon being passed, indicates that no more than an acceptable level of air remains within the balloon catheter 490.

It should be understood that the use of a motorized syringe system to perform the de-airing, as well as the measurement of vacuum pressure relationship to purge syringe displacement, may be used separately or together, with benefits resulting from each method. For example, the automation of the de-airing system simplifies the preparation of the balloon catheter 490, even if the final check of air in the system is performed manually with a visual check. Similarly, even if the de-airing is performed manually with syringes, the pressure vs. displacement measurements may still be utilized to eliminate the need to rely on a visual check to confirm that the balloon catheter 490 has been appropriately de-aired. Most preferably, the automation is combined with the pressure measurements to provide a simple and fast de-airing process that relies on objective measurements to determine when the balloon catheter 490 has been appropriately de-aired. Finally, it should be understood that, although the de-airing system described in connection with FIG. 7 is illustrated as a separate component, the system could be built into the handle 450 of the delivery system so as to not require a physically separate system to perform the de-airing procedure.

According to one aspect of the disclosure, a method of implanting a prosthetic heart valve comprises:
delivering the prosthetic heart valve to a native valve annulus while the prosthetic heart valve is crimped over a deflated balloon of a delivery device;
advancing fluid through the delivery device and into the balloon to inflate the balloon and to expand the prosthetic heart valve into the native valve annulus;
while advancing fluid through the delivery device, monitoring a pressure within the delivery device; and
displaying the monitored pressure on a display device in real time during monitoring the pressure; and/or
displaying the monitored pressure on the display device in real time includes displaying the monitored pressure as a function of an area of the balloon as a procedural curve; and/or
the area of the balloon is determined based on the monitored pressure, and the monitored pressure is determined by measuring pressure within a fluid line through which the fluid is advanced; and/or
the area of the balloon is estimated based on a volume of fluid advanced through the delivery device; and/or
the area of the balloon is determined based on a strain gauge mounted on the balloon; and/or
displaying on the display device a reference curve of pressure as function of balloon area; and/or
the reference curve is based on an expected relation of pressure of the balloon to area of the balloon when the balloon is being expanded in free space; and/or
monitoring a difference between the procedural curve and the reference curve while advancing fluid through the delivery device to expand the prosthetic heart valve; and/or
advancing fluid through the delivery device includes actuating an actuator on a handle of the delivery device; and/or
actuating the actuator on the handle of the delivery device sends a signal to a motorized housing having a syringe containing the fluid, the signal causing the motorized housing to depress a plunger of the syringe.

According to another aspect of the disclosure, a prosthetic heart valve delivery system comprises:
a handle;
a balloon catheter extending from the handle;
a balloon positioned at a distal end portion of the balloon catheter;
a fluid reservoir in fluid communication with a lumen, the lumen being in fluid communication with an interior volume of the balloon;
a motor operably coupled to the fluid reservoir; and
an actuator on the handle, the actuator being operably coupled to the motor so that actuation of the actuator in a first direction causes the motor to push fluid from the fluid reservoir through the lumen toward the interior volume of the balloon, and actuation of the actuator in a second direction opposite the first direction causes the motor to withdraw fluid from the interior volume of the balloon toward the fluid reservoir; and/or
the fluid reservoir and the motor are contained within the handle; and/or
the fluid reservoir is in a syringe coupled to a housing to receive the syringe, the motor being positioned within the housing; and/or
a pressure sensor configured to determine a pressure within the balloon; and/or the pressure sensor is positioned directly within the balloon; and/or
a strain gauge mounted on the balloon.

According to yet another aspect of the disclosure, a method of de-airing a balloon of a balloon catheter comprises:
(i) withdrawing a plunger of a purge syringe while the purge syringe is in fluid communication with the balloon of the balloon catheter and while the purge syringe is not in fluid communication with a fill syringe containing fluid, and monitoring vacuum pressure as a function of plunger displacement, as the plunger of the purge syringe is withdrawn, to create a pressure-displacement line having a slope;
(ii) advancing a plunger of the fill syringe while the fill syringe is in fluid communication with the balloon of the balloon catheter and while the fill syringe is not in fluid communication with the purge syringe;
(iii) advancing the plunger of the fill syringe while the fill syringe is in fluid communication with the purge syringe and while the fill syringe is not in fluid communication with the balloon catheter to push remaining air through a one-way valve in fluid communication with the purge syringe; and
repeating steps (i) through (iii) until the slope of the pressure-displacement line exceeds a threshold slope of a reference pressure-displacement line; and/or
the vacuum pressure is monitored using a pressure sensor in fluid communication with the purge syringe; and/or
the plunger of the fill syringe and the plunger of the purge syringe are each operable coupled to a motor system; and/or
the steps of withdrawing the plunger of the purge syringe and advancing the plunger of the fill syringe are performed automatically via the motor system; and/or
a three-way valve is positioned between the fill syringe, the purge syringe, and the balloon catheter, the three-way valve being actuatable to fluidly connect, at a given moment, any two of the fill syringe, the purge syringe, and the balloon catheter.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of implanting a prosthetic heart valve, the method comprising:
delivering the prosthetic heart valve to a native valve annulus while the prosthetic heart valve is crimped over a deflated balloon of a delivery device;
advancing fluid through the delivery device and into the balloon to inflate the balloon and to expand the prosthetic heart valve into the native valve annulus;
while advancing fluid through the delivery device, monitoring a pressure within the delivery device; and
displaying the monitored pressure on a display device in real time during monitoring the pressure.

2. The method of claim 1, wherein displaying the monitored pressure on the display device in real time includes displaying the monitored pressure as a function of an area of the balloon as a procedural curve.

3. The method of claim 2, wherein the area of the balloon is determined based on the monitored pressure, the monitored pressure being determined by measuring pressure within a fluid line through which the fluid is advanced.

4. The method of claim 2, wherein the area of the balloon is estimated based on a volume of fluid advanced through the delivery device.

5. The method of claim 2, wherein the area of the balloon is determined based on a strain gauge mounted on the balloon.

6. The method of claim 2, further comprising displaying on the display device a reference curve of pressure as function of balloon area.

7. The method of claim 6, wherein the reference curve is based on an expected relation of pressure of the balloon to area of the balloon when the balloon is being expanded in free space.

8. The method of claim 7, further comprising monitoring a difference between the procedural curve and the reference curve while advancing fluid through the delivery device to expand the prosthetic heart valve.

9. The method of claim 1, wherein advancing fluid through the delivery device includes actuating an actuator on a handle of the delivery device.

10. The method of claim 9, wherein actuating the actuator on the handle of the delivery device sends a signal to a motorized housing having a syringe containing the fluid, the signal causing the motorized housing to depress a plunger of the syringe.

11. A prosthetic heart valve delivery system comprising:
a handle;
a balloon catheter extending from the handle;
a balloon positioned at a distal end portion of the balloon catheter;
a fluid reservoir in fluid communication with a lumen, the lumen being in fluid communication with an interior volume of the balloon;
a motor operably coupled to the fluid reservoir; and
an actuator on the handle, the actuator being operably coupled to the motor so that actuation of the actuator in a first direction causes the motor to push fluid from the fluid reservoir through the lumen toward the interior volume of the balloon, and actuation of the actuator in a second direction opposite the first direction causes the motor to withdraw fluid from the interior volume of the balloon toward the fluid reservoir.

12. The prosthetic heart valve delivery system of claim 11, wherein the fluid reservoir and the motor are contained within the handle.

13. The prosthetic heart valve delivery system of claim 11, wherein the fluid reservoir is in a syringe coupled to a housing to receive the syringe, the motor being positioned within the housing.

14. The prosthetic heart valve delivery system of claim 11, further comprising a pressure sensor configured to determine a pressure within the balloon.

15. The prosthetic heart valve delivery system of claim 14, wherein the pressure sensor is positioned directly within the balloon.

16. The prosthetic heart valve delivery system of claim 14, further comprising a strain gauge mounted on the balloon.

17. A method of de-airing a balloon of a balloon catheter, the method comprising:
(i) withdrawing a plunger of a purge syringe while the purge syringe is in fluid communication with the balloon of the balloon catheter and while the purge syringe is not in fluid communication with a fill syringe containing fluid, and monitoring vacuum pressure as a function of plunger displacement, as the plunger of the purge syringe is withdrawn, to create a pressure-displacement line having a slope;
(ii) advancing a plunger of the fill syringe while the fill syringe is in fluid communication with the balloon of the balloon catheter and while the fill syringe is not in fluid communication with the purge syringe;
(iii) advancing the plunger of the fill syringe while the fill syringe is in fluid communication with the purge syringe and while the fill syringe is not in fluid communication with the balloon catheter to push remaining air through a one-way valve in fluid communication with the purge syringe; and
repeating steps (i) through (iii) until the slope of the pressure-displacement line exceeds a threshold slope of a reference pressure-displacement line.

18. The method of claim 17, wherein the vacuum pressure is monitored using a pressure sensor in fluid communication with the purge syringe.

19. The method of claim 18, wherein the plunger of the fill syringe and the plunger of the purge syringe are each operable coupled to a motor system.

20. The method of claim 19, wherein the steps of withdrawing the plunger of the purge syringe and advancing the plunger of the fill syringe are performed automatically via the motor system.

21. The method of claim 17, wherein a three-way valve is positioned between the fill syringe, the purge syringe, and the balloon catheter, the three-way valve being actuatable to fluidly connect, at a given moment, any two of the fill syringe, the purge syringe, and the balloon catheter.
